(19) United States
(12) Patent Application Publication
(10) Pub. No.: US 2024/0299721 A1
(43) Pub. Date: Sep. 12, 2024

(54) Catheter with Adhesion Detection

(71) Applicant: Bard Access Systems, Inc., Salt Lake City, UT (US)

(72) Inventors: David Colton Jackson, Cottonwood Heights, UT (US); Kenesaw M. Thornley, Millcreek, UT (US); Megan E. Brooks, Salt Lake City, UT (US)

(21) Appl. No.: 18/119,239

(22) Filed: Mar. 8, 2023

(51) Int. Cl. A61M 39/02; A61M 25/00

(52) U.S. Cl. CPC A61M 39/0247; A61M 25/0023; A61M 2039/0258; A61M 2039/0267; A61M 2205/3317; A61M 2205/502

(57) ABSTRACT

A catheter system including a vascular catheter having a catheter tube configured for placement within a patient body. A system module incorporated into the catheter tube adjacent the distal tip includes first and second electrodes disposed within the lumen and coupled with the luminal wall adjacent the distal tip. A console coupled with the first and second electrodes includes logic that determines an electrical impedance between the first and second electrodes, where the impedance is related to thrombus formation and/or the bacterial adhesion within the lumen. The system module provides a wireless notification when the impedance exceeds a defined limit. A secondary system includes a light activated surface coating applied to an inside luminal wall surface. Energizing a light source activates the surface coating to release an agent (e.g., an active pharmaceutical ingredient or an anti-coagulant agent) to reduce effects of the thrombus formation and/or the bacterial adhesion.

### BACKGROUND

Thrombus formation, bio-film build-up, and bacterial adhesions within the lumen of long term vascular catheters can cause reduced catheter function and catheter replacement at significant cost and risk to the patient. Current clinical practices can detect thrombus formation that may originate or be related to catheter related medical devices through various imaging and contrast modalities, such as Contrast Venographic imaging, duplex ultrasound, etc. However, these methods do not assess thrombus within the catheter directly and assessment cannot be made without complete removal of the device.

Catheter care teams and nurses can assess the presence of thrombus within the catheter during routine maintenance and administration by the presentation of reduced device functionality, such as pressure buildup or blockage, for example. This can lead to clinical judgement and intervention such as the administration of anticoagulants, e.g., low dose warfarin or low-molecular-weight heparin. However, typical thrombus formation must be present at an advanced stage to directly affect functionality of flow rates and injections in catheter related devices.

Central line related infections (CLABSI) and associated bloodstream infections (CRBSIs) can be difficult to identify, diagnose, and treat. A large protocol of external lab testing and bacterial culturing is required which can take extended amounts of time and lead to extended hospital stays. Clinical administration of antibiotics can treat blood related infection acutely but if the infection originated in the device, it can re-culture, proliferate, and cause reinfection. This leads to poor clinical outcomes, return hospital stays for the patient, etc. Additionally, hospitals are subjected to pay large fines for positive CLABSI cases, which are confirmed by lab testing where the tip of removed devices are cut and cultured in lab assays.

Disclosed herein are systems and methods that address the foregoing.

### SUMMARY

Disclosed herein is a catheter system that, according some embodiments, includes a catheter having a catheter tube configured for placement within a patient body, where the catheter tube includes a luminal wall that defines a lumen extending along the catheter tube between a hub and distal tip. The catheter system further includes a system module coupled with the catheter tube. The system module includes first and second electrodes disposed within the lumen, where the first and second electrodes are coupled with the luminal wall adjacent the distal tip, and where the second electrode is spaced away from the first electrode. The system module further includes a console coupled with the first and second electrodes, where the console includes a processor and a memory having logic stored thereon, and where the system module is configured for placement within the patient body during use. The logic, when executed by the processor, performs operations that include determining an electrical impedance between the first and second electrodes.

In some embodiments, the operations further include determining a first electrical impedance in accordance with a solution disposed within the lumen.

In some embodiments, determining an electrical impedance includes determining a change in electrical impedance away from the first electrical impedance, where the change in electrical impedance is caused by at least one of a thrombus formation or a bacterial adhesion within the lumen.

In some embodiments, the change in electrical impedance includes an increase in electrical impedance.

In some embodiments, the operations further include comparing a determined electrical impedance with an impedance limit stored in the memory, and as a result of the comparison, providing a notification when the determined electrical impedance exceeds the impedance limit.

In some embodiments, the console includes a wireless module, and providing a notification includes wirelessly transmitting the notification to an external computing device.

In some embodiments, the first electrode receives an excitation voltage from the console and the console receives a signal from the second electrode.

In some embodiments, at least a portion of each of the first and second electrodes is embedded within the luminal wall.

In some embodiments, the system module is disposed radially inward of an outside surface of the catheter tube. In some embodiments, the system module is disposed adjacent the distal tip. In some embodiments, the system module is embedded within the luminal wall.

In some embodiments, the catheter system further includes a secondary system including a light activated surface coating applied to an inside surface of the luminal wall, and the operations further include energizing a light source of the console to activate the surface coating, wherein activating the surface coating releases an agent configured to reduce the effects of the at least one of a thrombus formation or a bacterial adhesion within the lumen. In some embodiments, the agent includes at least one of an active pharmaceutical ingredient or an anti-coagulant agent.

Also disclosed herein is a method of the catheter system that, according to some embodiments, includes monitoring an electrical impedance between a pair of electrodes disposed within a lumen of a catheter of the catheter system. Where the catheter system includes a system module having a console operatively coupled with the pair of electrodes, and where the system module is coupled with the catheter such that the system module is disposed within a patient during use. The method further includes providing a notification when the electrical impedance exceeds a predefined impedance limit.

In some embodiments of the method, providing a notification includes wirelessly transmitting the notification to an external computing device.

In some embodiments of the method, a change in the electrical impedance is caused by at least one of a thrombus formation or a bacterial adhesion within the lumen.

In some embodiments of the method, monitoring an electrical impedance includes (i) determining a first electrical impedance in accordance with a solution disposed within the lumen and (ii) determining a change in the electrical impedance away from the first electrical impedance.

In some embodiments of the method, a system module of the catheter system is disposed within the patient. In some embodiments of the method, the system module is embedded within a luminal wall of the catheter adjacent the distal tip.

In some embodiments, the method further includes energizing a light source of the system module to activate a surface coating applied to an inside luminal wall surface, wherein activating the surface coating releases an agent configured to reduce effects of the at least one of a thrombus formation or a bacterial adhesion within the lumen. In some embodiments, the agent include at least one of an active pharmaceutical ingredient or an anti-coagulant agent.

These and other features of the concepts provided herein will become more apparent to those of skill in the art in view of the accompanying drawings and following description, which describe particular embodiments of such concepts in greater detail.

### DRAWINGS

FIG. 1 illustrates a catheter system, in accordance with some embodiments.

FIG. 2 is a detailed illustration of a distal portion of the catheter of the catheter system of FIG. 1, in accordance with some embodiments.

FIG. 3 is a perspective illustration of the system module of the catheter system of FIG. 1, in accordance with some embodiments.

FIG. 4A is a schematic illustration of the system module of the catheter system of FIG. 1 depicting a first instance of use, in accordance with some embodiments.

FIG. 4B is the schematic illustration of FIG. 4A depicting a second instance of use, in accordance with some embodiments.

FIG. 5 is a block diagram of a console of the system module of FIG. 3, in accordance with some embodiments.

FIG. 6 illustrates the catheter system of FIG. 1 further including a secondary system having a light activated surface coating, in accordance with some embodiments.

FIG. 7 illustrates a block diagram of a method of the catheter system, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to,” “coupled to/with,” and “in communication with” refer to any form of interaction between two or more entities, including but not limited to mechanical, electrical, magnetic, electromagnetic, fluid, and thermal interaction. Two components may be coupled with each other even though they are not in direct contact with each other. For example, two components may be coupled with each other through an intermediate component.

The terms “proximal” and “distal” refer to opposite ends of a medical device, including the devices disclosed herein. As used herein, the proximal portion of a medical device is the portion nearest a practitioner during use, while the distal portion is the portion at the opposite end. For example, the distal end of a catheter is defined as the end closest to the patient during utilization of the catheter, such as distal tip of the catheter, for example. The proximal end is the end opposite the distal end, such as extension legs of the catheter, for example.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Unless defined otherwise, all technical and scientific terms used herein have the same meaning as commonly understood by those of ordinary skill in the art.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method. Additionally, all embodiments disclosed herein are combinable and/or interchangeable unless stated otherwise or such combination or interchange would be contrary to the stated operability of either embodiment.

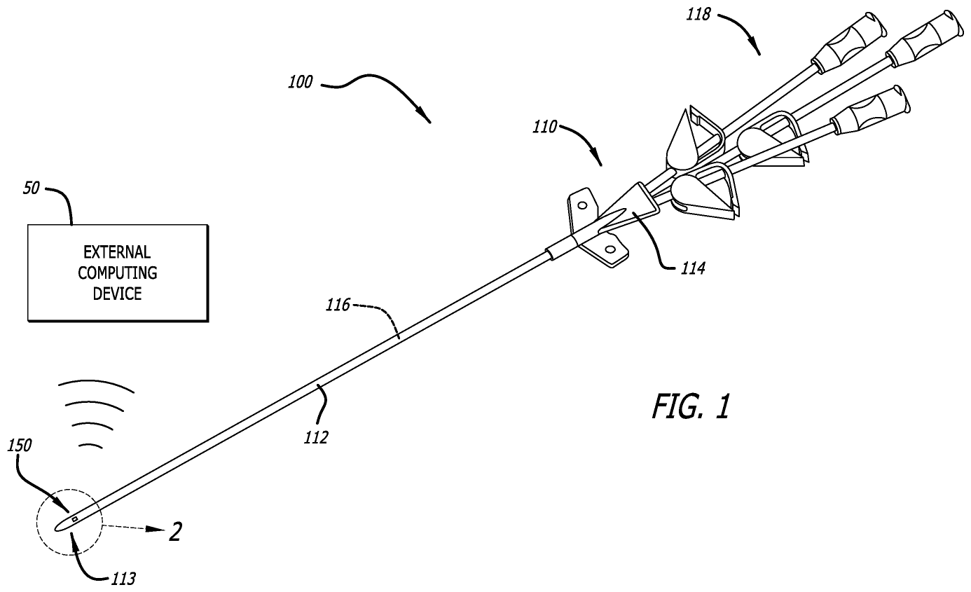

FIG. 1 illustrates a catheter system 100 that generally includes a catheter 110 and a system module 150 coupled with the catheter 110 adjacent a distal tip 113. The catheter 110 may generally include any tubular device configured for placement within a patient body, such as a central venous catheter (CVCs), peripherally inserted central catheter (PICC), a port infusion device, or a dialysis catheter, for example. Such catheters may be deployed with auxiliary equipment and tubing that contacts blood such as blood infusion equipment, plasma collection equipment, dialysis equipment, and the like. Foreign body ingress into the luminal space of a catheter, such as bacterial adhesion or thrombus formation, can affect the electrical impedance across/through the medium within the luminal space. This allows for an appropriate impedance measurement signal change that can be related to the bacterial adhesion or early thrombus formation.

The system module 150 is generally configured to monitor a condition of the catheter 110 such implicit monitoring of thrombus formation and/or bacterial adhesion directly at the inside surface of a lumen of the catheter 110, for example. This would allow for early detection and intervention by the clinician to improve catheter functionality and clinical outcomes. The burden and reliance on external lab testing and hospital infrastructure would be reduced. Furthermore, the necessity of catheter removal and complications of repeated catheter placement procedures may be mitigated. Monitoring at the direct site of infection and thrombus formation will arm the clinician with information to provide better care and outcomes for patients. In some embodiments, the system module 150 may be communicatively coupled with an external computing device 50. In some embodiments, the external computing device may be a cell phone, or a tablet, for example. In some embodiments, the external computing device 50 may associated with a healthcare facility network, or other auxiliary equipment.

The catheter 110 may include a catheter tube 112 configured for insertion within the patient body including a vasculature. The catheter tube 112 extends between a hub 114 and the distal tip 113. The catheter tube 112 includes a number (e.g., 1, 2, 3 or more) of lumens extending along the catheter tube 112 including the lumen 116. The catheter 110 also includes a number of extension legs 118 in fluid communication with the number of lumens. In some embodiments, the number of extension legs 118 may correspond to the number of lumens.

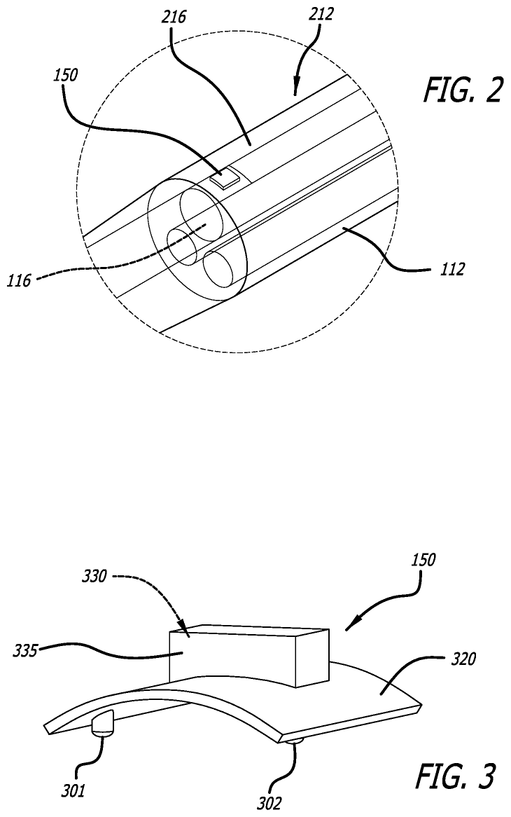

FIG. 2 is a detailed cross-sectional view of a distal portion of the catheter tube 112 adjacent the distal tip 113. Shown is the system module 150 operatively coupled with the lumen 116. In the illustrated embodiment, the system module 150 is embedded into the luminal 216 of the lumen 116. Consequently, the system module 150 is configured for placement within the patient body. The system module 150 may be disposed radially inward of an outside surface 212 of the catheter tube 112 so that the system module 150 may not interfere with placement of the catheter 110 within the patient body. A method of manufacturing the catheter 110 may include inserting molding the system module 150 into the luminal wall 216.

In the illustrated embodiment, the catheter system 100 includes a single system module 150 configured to monitor the condition of a single lumen. In other embodiments, the catheter system 100 may include more than one system module 150 to enable monitoring of more than one lumen. In similar fashion, the system module 150 may be configured to monitor the condition of more than one lumen.

FIG. 3 illustrates the system module 150 separated from the catheter tube 112. The system module 150 includes a first electrode 301 and a second electrode 302. The first and second electrodes 301, 302 are configured to extend into the lumen 116 (see FIG. 2) so that the first and second electrodes 301, 302 may electrically couple with a medium (e.g., a liquid solution) within the lumen 116. The first and second electrodes 301, 302 are spaced away from each other. In some embodiments, the first and second electrodes 301, 302 may be located on opposite sides of the lumen 116. The system module 150 may include a frame 320 to define a structure of the system module 150. In some embodiments, the frame 320 may include a printed circuit board. As shown, in some embodiments, the frame 320 may include a curved shape consistent with a curved shape of the luminal wall 216. The system module 150 includes a console 330 which may be contained within a housing 335 and the first and second electrodes 301, 302 are operatively coupled with the console 330. The system module 150 is generally configured to determine an electrical impedance between the first and second electrodes 301, 302 as further described below.

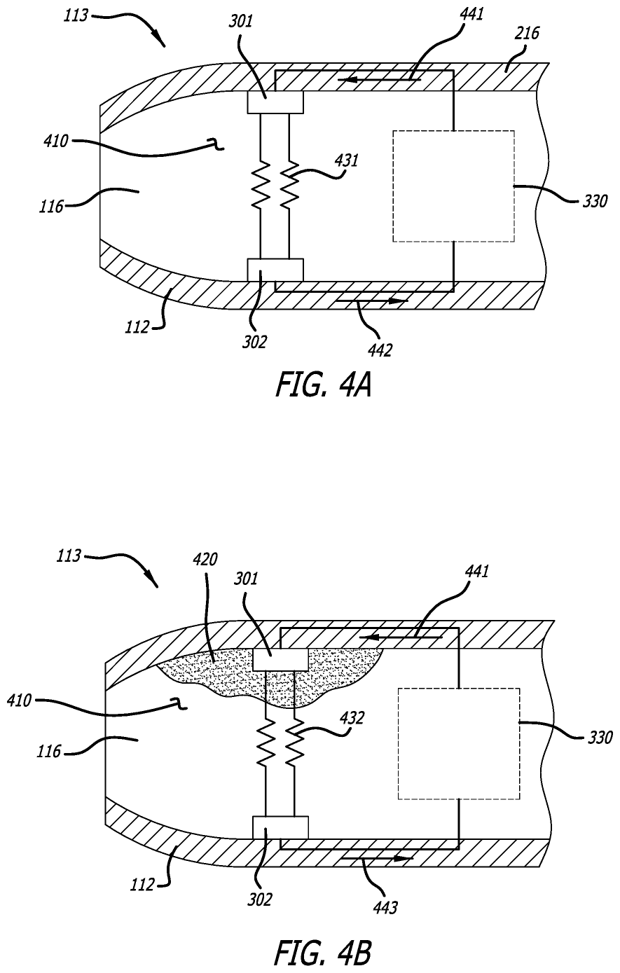

FIGS. 4A-4B are schematic illustrations of the system module 150 depicting the determination of the electrical impedance between the first and second electrodes 301, 302. In the illustrated embodiment, the electrical impedance may include a resistive component, a reactive component, and/or a combination thereof. Shown are the first and second electrodes 301, 302 electrically coupled with the console 330 and spaced across the lumen 116 from each other. In the illustrated embodiment, the console 330 provides an excitation voltage 441 to the first electrode 301 and a console 330 receives an electrical signal from the second electrode 302, where the electrical signal is related to an impedance between the first and second electrodes 301, 302.

FIG. 4A illustrates an instance where a solution 410 (e.g., saline) is disposed within the lumen 116, such as at the beginning of use of the catheter 110, for example. In such an instance, the base or first impedance 431 is related to the conductivity of the solution 410. In such an instance, the console 330 receives a first electrical signal 442 corresponding to the first impedance 431.

FIG. 4B illustrates another instance (e.g., subsequent instance) where a substance build-up 420, such as a bacterial adhesion or a thrombus formation, for example, is present within the lumen 116 along with the solution 410. In some instances, the substance build-up 420 may alter the impedance between the first and second electrodes 301, 302 defining a second impedance 432 different from the first impedance 431. In the illustrated example, the second impedance 432 may be greater than the first impedance 431. In such an instance, the console 330 receives a second electrical signal 443 corresponding to the second impedance 432.

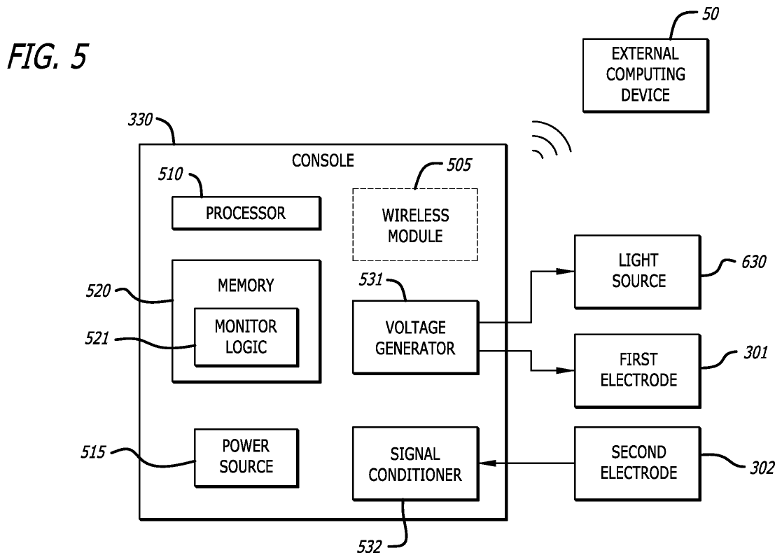

FIG. 5 illustrates a block diagram of the console 330, according to some embodiments. The console 330 is generally configured to govern the operation of the system module 150. The console 330 includes a processor 510 and memory 520 (e.g., a non-transitory computer-readable medium) having monitor logic 521 stored thereon. The console 330 may include a wireless module 505 to facilitate wireless communication with the external computing device 50. In some embodiments, the wireless module 505 may be omitted.

The console 330 includes a voltage generator 531 configured to define and deliver the excitation voltage 441 to the first electrode 301. A signal conditioner 532 receives the electrical signal from the second electrode 302 and converts electrical signal from the second electrode 302 to digital data for processing by the processor 510 according to the monitor logic 521.

The console 330 is powered via a power source 515. In some embodiments, the power source 515 may include an internal battery capable of providing sufficient power to operate the system module 150 over the life of the catheter system 100. Other power sources are also contemplated and therefore, include herein. For example, the catheter system 100 may include an external power source coupled with the console 330. In such embodiments, the catheter system 100 may include conductive elements extending along the catheter tube 112, such as wires embedded within the luminal wall 216, for example. In some embodiments, the one or more lumens containing a conductive solution may define one or more conductive elements coupling the external power source to the console 330.

In some embodiments, the console 330 may be disconnected from the power source 515 until a time of use of the catheter system 100. By way of one example, a second pair of electrodes may be disposed within a lumen (e.g., the lumen 116) where the second pair of electrodes are included in a power circuit of the console 330. The second pair of electrodes are configured such that the absence of a conductive solution within the lumen defines the power circuit in an open state defining a deactivated state of the console 330. Upon placement of a conductive solution within the lumen, the conductive solution electrically couples the second pair of electrodes together to define the power circuit in a closed state defining an activated state of the console 330.

The monitor logic 521 generally defines the operation of the system module 150 including the exemplary operations described below. The monitor logic 521 determines the impedance between the first and second electrodes 301, 302 based on the excitation voltage supplied to the first electrode 301 and the electrical signal received from the second electrode 302. The monitor logic 521 may determine the first impedance 431 during an instance where the solution 410 is disposed within the lumen, such as before the catheter 110 is inserted within the patient body, for example. The monitor logic 521 may record and store the first impedance 431 in the memory 520. The monitor logic 521 may monitor the impedance between the first and second electrodes 301, 302 during the use of the catheter 110 to detect presence of the build-up substance 420. In some embodiments, the monitor logic 521 may define an impedance limit, such as an increase of a defined amount above the first impedance 431, for example. The monitor logic 521 may then provide a notification when the monitored impedance exceeds the impedance limit. In some embodiments, the monitor logic 521 may wirelessly transmit the notification to the external computing device 50.

In some embodiments, the system module 150 may be transitionable between a standby mode and an operating mode, where the standby mode may be configured to conserve battery life. In some embodiments, the monitor logic 521 may receive a wireless signal from the external computing device 50 and as a result of receiving the wireless signal, the monitor logic 521 may transition the system module 150 from the standby mode to the operating mode.

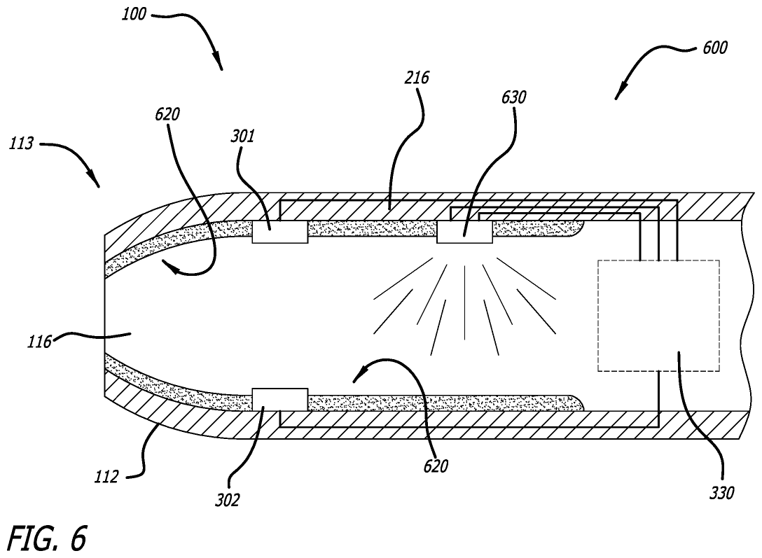

FIG. 6 is schematic illustration of the catheter system 100 further including a secondary system 600, according some embodiments. In some embodiments, the secondary system 600 may be omitted. The secondary system 600 includes a coating 620 disposed on an inside surface of the luminal wall 216 along at least a distal portion of the catheter tube 112 adjacent the distal tip 113. The coating 620 is configured to transition from a passive state to an active state when exposed to a light. In the illustrated embodiment, the secondary system 600 includes a number (e.g., 1, 2, 3, or more) of light sources 630, such as light emitting diodes (LEDs), for example, operatively coupled with the console 330. As such, the monitor logic 221 may be configured to energize the light sources 630 and thereby, activate the coating 620. In some embodiments, the monitor logic 221 may energize the light sources 620 proactively as a preventative measure against the substance build-up 420 (FIG. 4B). In some embodiments, the monitor logic 221 may energize the light source(s) 630 in response to an increase in the monitored impedance, such as when the monitored impedance exceeds a defined impedance limit, for example.

The coating 620 is generally configured to reduce the effects of the substance build-up 420 (FIG. 4B) when activated. In some embodiments, the coating 620 may release a substance configured to denature (i.e., kill, disrupt, remove, etc.) the substance build-up 420. For example, in some embodiments, the coating 620 may release an active pharmaceutical agent. In other embodiments, the coating 620 may release an anti-coagulant agent.

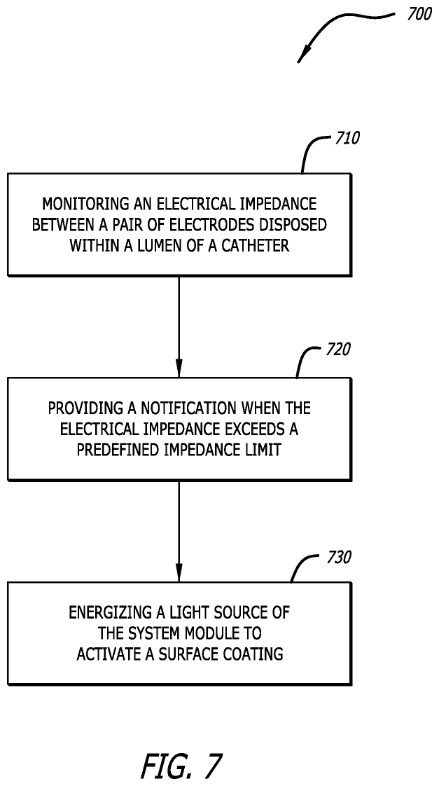

FIG. 7 illustrates a block diagram of a method 700 of the catheter system that, according to some embodiments, may include all or any subset of the following steps, actions, or processes. The method 700 includes monitoring an electrical impedance between a pair of electrodes disposed within a lumen of a catheter of the catheter system (block 710). The pair of electrodes are disposed adjacent the distal tip of the catheter. A change in the electrical impedance may be caused by a thrombus formation, a bacterial adhesion, or the like within the lumen. Monitoring the electrical impedance may include (i) determining a first electrical impedance in accordance with a solution disposed within the lumen (e.g., saline) and (ii) determining a change in the electrical impedance away from the first electrical impedance.

The method 700 may further include providing a notification when the electrical impedance exceeds a predefined impedance limit (block 720). Providing the notification may include wirelessly transmitting the notification to an external computing device.

The method 700 may further include energizing a light source of the system module to activate a surface coating (block 730), where the surface coating may be applied to an inside luminal wall surface of the catheter tube. Activating the surface coating may release an agent configured to reduce effects of the at least one of a thrombus formation or a bacterial adhesion within the lumen. The agent include at least one of an active pharmaceutical ingredient or an anti-coagulant agent.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A catheter system, comprising:
a catheter comprising:
a catheter tube configured for placement within a patient body; and
a luminal wall defining a lumen extending along the catheter tube between a hub and distal tip; and
a system module coupled with the catheter, comprising:
first and second electrodes disposed within the lumen, wherein:
the first and second electrodes are coupled with the luminal wall adjacent the distal tip, and
the second electrode spaced away from the first electrode; and
a console coupled with the first and second electrodes, the console including a processor and a memory having logic stored thereon that, when executed by the processor, performs operations that include determining an electrical impedance between the first and second electrodes,
wherein the system module is configured for placement within the patient body.

2. The system according to claim 1, wherein the change in the electrical impedance is caused by at least one of a thrombus formation or a bacterial adhesion within the lumen.

3. The system according to claim 1, wherein the operations include determining a first electrical impedance in accordance with a solution disposed within the lumen.

4. The system according to claim 3, wherein determining an electrical impedance includes determining a change in electrical impedance away from the first electrical impedance.

5. The system according to claim 4, wherein the change in electrical impedance includes an increase in electrical impedance.

6. The system according to claim 1, wherein the operations further include:
comparing a determined electrical impedance with an impedance limit stored in the memory; and
as a result of the comparison, providing a notification when the determined electrical impedance exceeds the impedance limit.

7. The system according to claim 6, wherein:
the console includes a wireless module, and
providing a notification includes wirelessly providing the notification to an external computing device.

8. The system according to claim 1, wherein:
the first electrode receives an excitation voltage from the console, and
the console receives a signal from the second electrode.

9. The system according to claim 1, wherein at least a portion of each the first and second electrodes is embedded within the luminal wall.

10. The system according to claim 1, wherein the system module is disposed radially inward of an outside surface of the catheter tube.

11. The system according to claim 1, wherein the system module is disposed adjacent the distal tip.

12. The system according to claim 1, wherein the system module is embedded within the luminal wall.

13. The system according to claim 2, further comprising a secondary system including a light activated surface coating applied to an inside surface of the luminal wall, the operations further including energizing a light source of the console to activate the surface coating, wherein activating the surface coating releases an agent configured to reduce effects of the at least one of a thrombus formation or a bacterial adhesion within the lumen.

14. The system according to claim 13, where in the agent includes at least one of an active pharmaceutical ingredient or an anti-coagulant agent.

15. A method of a catheter system, comprising:
monitoring an electrical impedance between a pair of electrodes disposed within a lumen of a catheter of the catheter system, wherein:
the catheter system includes a system module having a console operatively coupled with the pair of electrodes, and
the system module is coupled with the catheter such that the system module is disposed within a patient during use; and
providing a notification when the electrical impedance exceeds a predefined impedance limit.

16. The method according to claim 15, wherein a change in the electrical impedance is caused by at least one of a thrombus formation or a bacterial adhesion within the lumen.

17. The method according to claim 15, wherein monitoring an electrical impedance includes:
determining a first electrical impedance in accordance with a solution disposed within the lumen; and
determining a change in the electrical impedance away from the first electrical impedance.

18. The method according to claim 15, wherein providing a notification includes wirelessly transmitting the notification to an external computing device.

19. The method according to claim 16, further comprising energizing a light source of the system module to activate a surface coating applied to an inside luminal wall surface, wherein activating the surface coating releases an agent configured to reduce effects of the at least one of a thrombus formation or a bacterial adhesion within the lumen.

20. The method according to claim 19, where in the agent includes at least one of an active pharmaceutical ingredient or an anti-coagulant agent.
